(19) United States
(12) Patent Application Publication
(10) Pub. No.: US 2013/0302329 A1
(43) Pub. Date: Nov. 14, 2013

(54) DIAGNOSTIC TESTS FOR IMMUNE REACTIVITY WITH HUMAN ENDOTHELIAL CELL GROWTH FACTOR

(71) Applicants: Allen C. Steere, Wellesley, MA (US); Elise Drouin, Stoneham, MA (US); Catherine Costello, Reading, MA (US); Robert Seward, Malden, MA (US)

(72) Inventors: Allen C. Steere, Wellesley, MA (US); Elise Drouin, Stoneham, MA (US); Catherine Costello, Reading, MA (US); Robert Seward, Malden, MA (US)

(73) Assignees: TRUSTEES OF BOSTON UNIVERSITY, Boston, MA (US); THE GENERAL HOSPITAL CORPORATION, Boston, MA (US)

(21) Appl. No.: 13/823,126

(22) PCT Filed: Sep. 21, 2011
(86) PCT No.: PCT/US11/52456

§ 371 Date: May 24, 2013

Related U.S. Application Data
(60) Provisional application No. 61/384,733, filed on Sep. 21, 2010.

(51) Int. Cl. G01N 33/68

(52) U.S. Cl. CPC G01N 33/6863
USPC 424/134.1; 435/7.1; 435/7.92; 424/142.1; 424/158.1; 435/7.94; 514/313; 514/249

(57) ABSTRACT

The present invention provides for methods and compositions for identifying and detecting autoantigens. Candidate autoantigens are identified by obtaining a subject sample from which HLA-DR-presented peptides are collected and identified using mass spectometry, then synthesized and reacted with the same subject peripheral blood or effected tissue. In particular, the present invention provides for endothelial cell growth factor as a novel autoantigen biomarker for Lyme disease-associated arthritis.

### CROSS REFERENCE TO RELATED APPLICATIONS

This application claims the benefit of U.S. Provisional Application No. 61/348,733, filed Sep. 21, 2010, and incorporated fully herein by this reference.

### FEDERAL FUNDING

This invention was made with Federal funding under grants AR-20358, P41 RR10888, S10 RR15942, S10 RR20946 and Contract N01 HV28178, awarded by the National Institutes of Health. The U.S. Government has certain rights in the invention.

### FIELD OF THE INVENTION

The present invention provides for methods and compositions for identifying and detecting humoral and cellular autoimmune responses to disease-related autoantigens. In particular, the present invention provides for a novel autoantigenic biomarker, endothelial cell growth factor, for Lyme disease-associated arthritis.

### BACKGROUND

Lyme disease, or borreliosis, is the most common vector-borne infectious disease in North America, Europe and Asia. Lyme disease has major public health and economic effects: the estimated annual cost is approximately $1 billion in the U.S. alone. State health departments reported about 30,000 confirmed cases and 8,500 probable cases of Lyme disease to the Centers for Disease Control and Prevention in 2009, representing a 3.6 percent increase in confirmed cases compared to the previous year.

Caused by the spirochete bacterium Borrelia, and transmitted to humans through the bite of infected ticks, Lyme disease is a multi-system disorder that is treatable with antibiotics and can affect the nervous system, heart, and in particular the joints. Some patients develop chronic Lyme disease, a condition characterized by persistent musculoskeletal and peripheral nerve pain, fatigue, and memory impairment. In subjects with joint involvement, a small percentage develop proliferative synovitis that persists for months or several years after apparent spirochetal killing with antibiotics, referred to as antibiotic-refractory arthritis. This disease course is hypothesized to result from infection-induced autoimmunity, however none of the previously proposed candidate autoantigens induced substantial T- and B-cell responses in antibiotic-refractory Lyme arthritis patients. Hence, there remains a need in the art for characterization of the autoantigen(s) implicated in Lyme arthritis patients.

### SUMMARY

The embodiments of the present invention provide for novel biomarker autoantigen(s), platelet-derived endothelial cell growth factor (ECGF), useful for diagnosing Lyme arthritis. More specifically, tandem mass spectrometry identified HLA-DR self-peptides presented in vivo from subjects' synovial tissue, the target tissue in this disease. Of 120 peptides identified from one patient, one peptide, which originated from the source protein platelet-derived endothelial cell growth factor (ECGF), induced peripheral blood mononuclear cells (PBMC) to proliferate and secrete IFN-γ in vitro. It was then shown that many patients with antibiotic-refractory arthritis had T-cell responses to ECGF peptides and autoantibodies to this self protein. Furthermore, the majority of patients in the refractory group had greater amounts of ECGF in joint fluid and synovial tissue, where it could be presented by HLA-DR molecules on local antigen-presenting cell. In addition, IgG anti-ECGF antibodies were found predominately in Lyme disease, and were present significantly more often in those subjects with antibiotic-refractory arthritis. Thus, ECGF is the first autoantigen identified that induces both T- and B-cell responses in subjects with antibiotic-refractory Lyme arthritis. The methods and compositions of the present embodiments should aid clinicians in designing better therapeutic approaches in treating subjects suffering from chronic inflammatory arthritis. Moreover, the present methodology, that combines discovery-based proteomics and translational research, is applicable to other autoimmune diseases where identifying pathogenic autoimmune responses has been a difficult challenge.

An embodiment of the present invention provides for a method for determining whether a biological sample obtained from a subject is reactive with endothelial cell growth factor (ECGF) autoantigen comprising contacting said biological sample with an immunoassay comprising at least one ECGF epitope. The immunoassay may identify the presence of ECGF autoantigen-binding antibodies in the serum of a subject, such immunoassays may be an ELISA, agglutination test, direct immunofluorescence assay, indirect immunofluorescence assay, western blot, an immunoblot assay, and the like. Alternatively, the immunoassay may be a T-cell proliferation assay such as ³H-thymidine incorporation, CFSE dilution, etc. The immunoassay may be a T-cell reactivity assay; for example, an immunoassay that comprises measuring secretion of IFN-γ from individual cells, e.g., an ELISpot immunoassay. In particular, the ECGF autoantigen used for such assays may consist of the intact protein, protein fragments or specific ECGF peptides, all with or without modifications. These peptides may include, but are not limited to, at least one ECGF peptide selected from the group consisting of: LGRFERMLAAQGVDPG (SEQ ID NO:1); ADIRGFVAAVVNGSAQGAQI (SEQ ID NO:2); DKVSLVLAPALAACG (SEQ ID NO:3); SKKLVEGLSALVVDV (SEQ ID NO:4); KTLVGVGASLGLRVAAALTAMD (SEQ ID NO:5); LRDLVTTLGGALLWL (SEQ ID NO:6); GTVELVRALPLALVLH (SEQ ID NO:7); or a functionally equivalent analog or derivative of thereof. The biological sample may be obtained from peripheral blood (e.g., serum), synovial fluid, synovial tissue, peripheral blood mononuclear cells (PBMC), or synovial fluid mononuclear cells (SFMC). The biological sample may be obtained from a subject suffering from chronic, inflammatory arthritis. In another aspect, a positive result of immunoreactivity of the biological sample with the ECGF autoantigen is indicative of Lyme arthritis, particularly antibiotic-refractory Lyme arthritis.

Another embodiment of the invention provides for the use of an isolated ECGF autoantigen as a biomarker for diagnosing Lyme arthritis. The isolated ECGF autoantigen can be human ECGF intact protein, protein fragments or specific ECGF peptides, all with or without modifications; or a functionally equivalent analog or derivative of thereof; or may include, but not limited to at least one of the following peptides: LGRFERMLAAQGVDPG (SEQ ID NO:1); ADIRGFVAAVVNGSAQGAQI (SEQ ID NO:2); DKVSLVLAPALAACG (SEQ ID NO:3); SKKLVEGLSALVVDV (SEQ ID NO:4); KTLVGVGASLGLRVAAALTAMD (SEQ ID NO:5); LRDLVTTLGGALLWL (SEQ ID NO:6); GTVELVRALPLALVLH (SEQ ID NO:7); or a functionally equivalent analog or derivative of thereof.

An embodiment of the present invention provides for a method of identifying autoantigens associated with autoimmune disorders. A biological sample is obtained from the subject, for example, blood, synovial fluid, or synovial tissue; HLA-DR-presented peptides are eluted from the sample; eluted peptides are identified, for example by mass spectrometry; corresponding peptides are synthesized, and; synthesized peptides are reacted with a biological sample obtained from the same subject, for example peripheral blood or synovial fluid mononuclear cells; whereby the in vitro reaction (i.e., a change in the sample) is characterized as an indicator of disease state.

A specific embodiment provides for a method for determining whether a subject suffering from chronic inflammatory arthritis bears T-cells reactive to ECGF or ECGF peptides by (a) providing a set of synthesized ECGF peptides or epitopes that are predicted to be presented by HLA-DR molecules; (b) stimulating peripheral blood mononuclear cells (PBMC) or the synovial fluid mononuclear cells (SFMC) obtained from the subject with one of said ECGF peptides or epitopes; and (c) measuring T-cell proliferation in vitro or secretion of IFN-γ as a test for T-cell reactivity. The subject can be suffering from either antibiotic-refractory or antibiotic-responsive Lyme arthritis.

Another specific embodiment provides for a method for determining whether a subject suffering from chronic inflammatory arthritis contains B-cell antibody against ECGF by (a) providing an isolated antigen, wherein said antigen is ECGF, or a functionally equivalent peptide, analog or derivative thereof; (b) providing a serum sample from a subject; (c) conducting an immunoassay on said sample utilizing said antigen, wherein said immunoassay detects the presence of antibodies that recognize said ECGF; and (d) determining that the subject contains an antibody against said antigen if the results of the immunoassay indicate that an antibody that recognizes said antigen is present in said sample. The subject can be suffering from either antibiotic-refractory or antibiotic-responsive Lyme arthritis. The immunoassay may comprise an ELISA, agglutination test, direct immunofluorescence assay, indirect immunofluorescence assay, western blot, an immunoblot assay, and the like.

Yet another embodiment is directed to a kit for identifying a subject with chronic inflammatory arthritis as having Lyme arthritis, comprising ECGF or a set of synthesized ECGF peptides/epitopes, and reagents necessary for conducting an immunoassay, wherein the immunoassay is capable of detecting the presence of an antibody in a sample, and wherein the antibody is capable of binding to said antigen. The subject can be suffering from either antibiotic-refractory or antibiotic-responsive Lyme arthritis. The immunoassay may comprise an ELISA, agglutination test, direct immunofluorescence assay, indirect immunofluorescence assay, western blot, an immunoblot assay, and the like.

Yet another embodiment is directed to a kit for idenifying a subject with chronic inflammatory arthritis as having Lyme arthritis, comprising ECGF or a set of synthesized ECGF peptides/epitopes and reagents necessary for conducting an immunoassay, wherein the immunoassay is capable of detecting the presence of T cell responses to said antigen in patients' biological samples. The subject can be suffering from either antibiotic-refractory or antibiotic-responsive Lyme arthritis. The immunoassay may comprise, but not limited to, ³H-thymidine incorporation assay, CFSE dilution, ELISpot, and the like.

The ECGF, ECGF peptides and/or ECGF epitopes of the present invention can contain naturally occurring amino acids, or can contain derivatives or analogs or combinations thereof. In a particular aspect, an ECGF peptide comprising, for example, the amino acid sequences LGRFERMLAAQGVDPG (SEQ ID NO:1); ADIRGFVAAVVNGSAQGAQI (SEQ ID NO:2); DKVSLVLAPALAACG (SEQ ID NO:3); SKKLVEGLSALVVDV (SEQ ID NO:4); KTLVGVGASLGLRVAAALTAMD (SEQ ID NO:5); LRDLVTTLGGALLWL (SEQ ID NO:6); GTVELVRALPLALVLH (SEQ ID NO:7); or a functionally equivalent derivative or analog thereof that binds with antibodies and/or stimulates a T-cell reaction in a sample obtained from a subject suffering from Lyme arthritis, particularly antibiotic-refractory Lyme arthritis.

### DESCRIPTION OF THE DRAWINGS

FIG. 1 is an overview of the isolation and identification of in vivo HLA-DR presented peptides from patients' synovial tissue. Antibiotic-refractory Lyme arthritis usually manifests as one swollen knee (1a). In those cases in which joint swelling persists after >3 months of oral and intravenous antibiotics followed by a suboptimal response to disease-modifying antirheumatic drugs (DMARD), therapeutic arthroscopic synovectomies are sometimes performed. During the procedure, 20 g to 40 g of inflamed synovial tissue and subcutaneous fat are removed (1b). Immunohistologic staining of the synovial tissue shows marked exogenous expression of HLA-DR molecules (1c). The tissue is separated from the fat and HLA-DR complexes are immunoprecipitated from synovial cell lysates using a HLA-DR specific antibody (1d). HLA-DR presented peptides are eluted and identified by tandem mass spectrometry. In this case, the LC/MS/MS spectra of the ECGF₃₄₀₋₃₅₅ peptide are shown (1e).

FIG. 2 is a bar graph of data from the screening of 120 HLA-DR-presented peptides identified from the synovial tissue of one patient for T cell autoantigenicity using the patient's own T cells. PBMC obtained near the time of synovectomy were washed and resuspended in 200 μl of complete medium at a concentration of 2×10⁵ cells per well. All non-redundant HLA-DR presented peptides identified from the patient's synovial tissue sample were synthesized and tested in sets of three (2 μM of each peptide). After incubation for 5 days at 37° C. and 5% CO₂, 0.5 μCurie of ³H-thymidine was added to each well, and cells were harvested 18 hours later to measure incorporation of ³H-thymidine into DNA. The data is presented as the average counts per minute (CPM) of duplicate wells for each peptide set (3 peptides/set, x-axis). A positive result was defined as a proliferative response>2 times background.

FIG. 3 shows data testing PBMC from patients with antibiotic-refractory or antibiotic-responsive Lyme arthritis, RA or healthy control subjects for T-cell recognition of ECGF peptides. Cells of patients with Lyme arthritis were collected from patients seen over the past 12 years. PBMC were plated as described in FIG. 2 and individual ECGF peptides (1 μM) were added to duplicate wells. After 5 days, cells were transferred to ELISpot plates previously coated with an IFN-γ capture antibody, and the assay was performed following the manufacturer's instructions (MabTech, Ohio, USA). Seven ECGF peptides, five of which were predicted to be promiscuous HLA-DR binders (binding more than 19 HLA-DR molecules, indicated in bold) were tested and the sequences were as follows: ⁵²ADIRGFVAAVVNGSAQGAQI⁷¹ (SEQ ID NO:2); ¹²³DKVSLVLAPALAACG₁³⁷ (SEQ ID NO:3); ²²⁰SKKLVEGLSALVVDV²³⁴ (SEQ ID NO:4); ²⁵³KTLVGVGASLGLRVAAALTAMD²⁷⁴ (SEQ ID NO:5); ³⁴⁰LGRFERMLAAQGVDPG³⁵⁵ (SEQ ID NO:1) (this peptide, which is circled in red, was identified in LA1 synovial sample); ³⁰²LRDLVTTLGGALLWL³¹⁶ (SEQ ID NO:6); and ³⁸⁷GTVELVRALPLALVLH⁴⁰¹ (SEQ ID NO:7). The five peptides predicted to be promiscuous binders were tested in all patients or control subjects, whereas the 2 non-promiscuous binders were tested in only a subset of patient or control cells (i.e., 15 of 18 healthy control subjects, none of the 12 RA patients, 18 of 19 patients with EM, 7 of 28 antibiotic-responsive arthritis patients, and 12 of 38 antibiotic-refractory arthritis patients) due to limited availability of cells.

FIG. 4 shows IgG anti-ECGF autoantibody responses in the sera of patients with various manifestation of Lyme disease, RA or healthy control subjects. Cells of patients with Lyme disease were collected from patients seen over the past 25 years. IgG anti-ECGF autoantibodies in serum samples were determined by both ELISA and immunoblotting. See Examples, below for details.

FIG. 5 shows detection of ECGF protein in the synovial fluid of antibiotic-responsive, antibiotic-refractory and the synovial tissue of an antibiotic-refractory Lyme arthritis patient. (5a) reflects ECGF concentrations in synovial fluid measured by sandwich ELISA. (5b) Representative serial synovial tissue sections from an antibiotic-refractory Lyme arthritis patient stained with anti-ECGF or isotype control antibodies. Immunohistochemical staining of ECGF was moderate to intense in both the synovial lining and sublining of antibiotic-refractory Lyme arthritis patients (20×). At a higher magnification (400×), positive ECGF staining can be observed in the cytoplasm and nucleus of fibroblast-like cells in the lining (arrow). Intense staining of ECGF was seen in areas surrounding microvessels (circled) in synoival sublining.

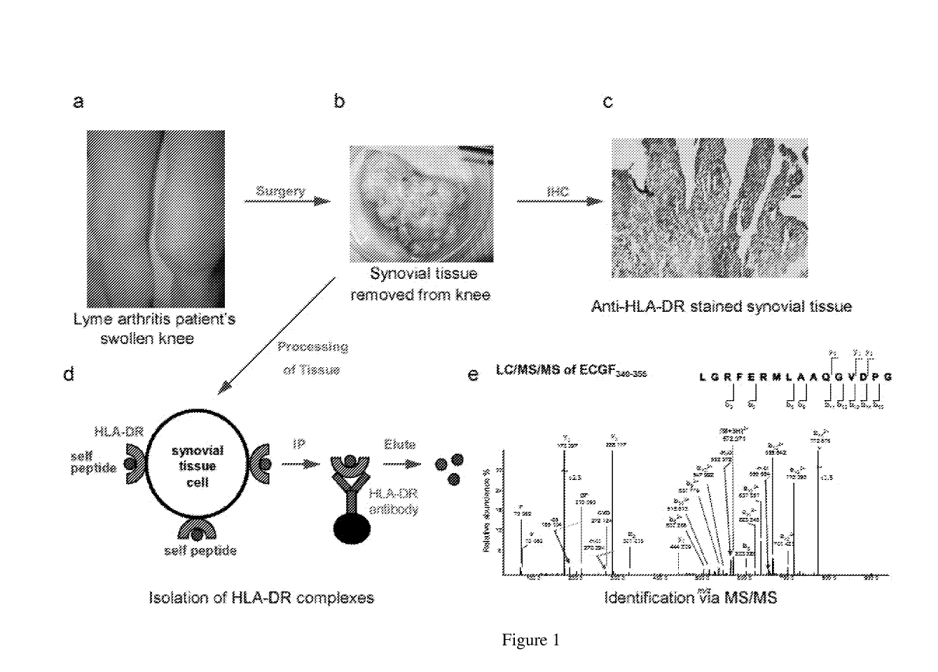

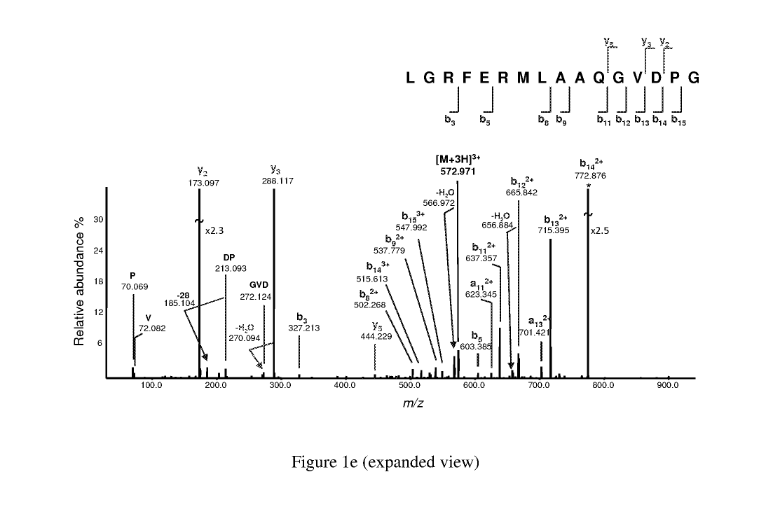

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Antibiotic-refractory Lyme arthritis, defined as persistent synovitis for months to years after spirochetal killing with antibiotic therapy, is hypothesized to result from B. burgdorferi-induced autoimmunity. Lyme borreliosis, the most common tick-borne disease in the northern hemisphere, results from infection with spirochetes of the Borrelia burgdorferi sensu lato complex. In the U.S., Borrelia burgdorferi sensu stricto (Bb) is the sole cause of the illness, whereas in Europe, Borrelia afzelii (Ba) and Borrelia garinii (Bg) are the primary pathogens. Steere, 345 N. Engl. J. Med. 115 (2001); Baranton et al., 42 Intl. J. Syst. Bacteriol. 378 (1992); Canica et al., 25 Scand. J. Infect. Dis. 441 (1993).

Infection with each of these species usually begins with a slowly expanding skin lesion, called erythema migrans (EM), which occurs at the site of the tick bite. In the U.S., where Bb is particularly arthritogenic, about 60% of un-treated patients develop intermittent or chronic joint swelling in later months, especially affecting knees. Steere et al., 107 Annals Internal Med. 725 (1987). These patients can usually be treated successfully with 1 month of oral or intravenous (IV) antibiotics, an outcome called antibiotic-responsive arthritis. Steere et al., 37 Arthritis Rheum. 878 (1994); Dattwyler et al., 117 Wiener klinische Wochenschrift 393 (2005). In a small percentage of patients, however, proliferative synovitis persists for months or several years after apparent spirochetal killing with >2 months of oral antibiotics, >1 month of intravenous antibiotics, or usually both, referred to as antibiotic-refractory arthritis. Steere & Angelis, 54 Arthritis Rheum. 3079 (2006a). This disease course is hypothesized to result from infection-induced autoimmunity.

Evidence supporting the hypothesis that Lyme arthritis is an infection-induced autoimmunity disease is 3-fold: (1) PCR and culture results of synovectomy specimens have been uniformly negative (Nocton et al., 330 New. Engl. J. Med. 229 (1994)); (2) relapse of infection has not been observed with the use of DMARDs after antibiotic therapy (Steere & Angelis, 2006a); and (3) specific HLA-DR alleles are the greatest known genetic risk factor for antibiotic-refractory arthritis⁹, a risk factor commonly associated with autoimmune diseases. Steere et al., 203 J. Experi. Med. 961 (2006b).

A number of spirochetal and host risk factors associated with antibiotic-refractory Lyme arthritis. These factors include infection with highly inflammatory B. burgdorferi RST1 (OspC type A) strains; a host Toll-like receptor 1 (TLR1) polymorphism (1805GG) which leads to exceptionally high inflammatory responses; certain HLA-DR molecules, such as the DRB1*0401 molecule, that bind an epitope of B. burgdorferi outer-surface protein A (OspA₁₆₅₋₁₇₃); high levels of inflammatory cytokines and chemokines in joint fluid, particularly CXCL9 and CXCL10, which are chemoattractants for T_{H}1 effector cells; and a dominant T_{H}1 response in joint fluid with persistently low percentages of Treg. The present invention adds to these factors a biomarker, the autoantigen ECGF, which is present and induces autoreactive immune cells in many, but not all, antibiotic-refractory Lyme arthritis patients.

As with all forms of chronic inflammatory arthritis, including RA, the synovial lesion in in patients with antibiotic-refractory arthritis shows synovial hypertrophy, vascular proliferation, infiltration of mononuclear cells, and intense expression of human leukocyte antigen D-related (HLA-DR) molecules. In antibiotic-refractory Lyme arthritis, the HLA-DR risk alleles include HLA-DRB1*0101, 0401, 0404, 0405 and 1501/DRB5*0101 (Steere et al., 2006b), similar to those in rheumatoid arthritis (RA) (Deighton et al., 36 Clin. Genet. 178 (1989); Seldin et al., 42 Arthritis Rheum. 1071 (1999)). HLA-DR molecules present peptides, both foreign and self, to CD4+ T-cells, which leads to T-cell activation and proliferation. With tissue-specific autoimmune diseases, HLA-DR molecules in the target tissue, in this case synovial tissue, are thought to present disease-related autoantigenic epitopes, but for the most part these epitopes have not yet been identified.

Initial attempts to uncover autoantigens in antibiotic-refractory Lyme arthritis were based upon a search for molecular mimicry between an epitope of Bb outer-surface protein A (OspA₁₆₃₋₁₇₅), which is bound by refractory arthritis-associated HLA-DR molecules, and those in human proteins. Steere et al., 52 Clin. Inf. Dis. S259 (2011). The first candidate autoantigen identified was LFA-1α_{L332-340} (Gross et al., 281 Sci. 703 (1998)), which has sequence homology with six of the nine core amino acid residues in OspA₁₆₃₋₁₇₅; and later, MAWD-BP₂₈₀₋₂₈₈ which was shown to have sequence identity with eight of the nine core OspA residues. Drouin et al., 45 Molec. Immunol. 108 (2008). Only a minority of patients had T-cell reactivity with these peptides, however, and none had B-cell responses to these self proteins. Id.; Steere et al., 48 Arthritis Rheum. 534 (2003). Others, using recombinant antibody probes derived from B-cells in patients' synovial lesions, identified cytokeratin 10 as a candidate autoantigen. Ghosh et al., 117 J. Immunol. 2486 (2006). This probe cross-reacted with OspA, but only three of fifteen patients with antibiotic-refractory arthritis had slight antibody responses to this self protein. Hence, none of these known candidate autoantigens appeared to explain antibiotic-refractory Lyme arthritis.

The identification of in vivo HLA-DR-presented peptides in synovial tissue was developed as a novel, unbiased approach that combines discovery-based proteomics with translational research for the identification of immunogenic self antigens. This protocol has steps comprising: (a) a proteomics approach utilizing tandem mass spectrometry (MS/MS) for the identification of HLA-DR-presented peptides in individual subject's synovial tissue; (b) synthesis and testing of all peptides identified in step (a) for reactivity with the same subject's PBMC, thereby asking the subject's own T-cells to indicate which HLA-DR self-peptides may be acting as autoantigens; and (c) validation that any immunogenic peptides and their source proteins identified in a single subject in step B also induce T- and B-cell reactivity in large numbers of subjects with Lyme arthritis.

The initial results based on mass spectrometry analysis of HLA-DR-eluted peptides from synovial tissue from four patients, two with antibiotic-refractory arthritis and two with RA were reported in Seward et al., 10 Molec. Cell Proteomics M110 002477 (2011). This approach identified 1,427 synovial HLA-DR in vivo-presented peptides (220 to 464 per patient), which on average represents a ten-fold increase in peptide identification compared with four previous studies of other tissues or fluids. See Gordon et al., 25 Eur. J. Immunol. 1473 (1995); Muixi et al., 181 J. Immunol. 785 (2008); Oshitani et al., Intl. J. Molec. Med. 99 (2003); Wahlstrom et al., 117 J. Clin. Investig. 3576 (2007). The 1,427 peptides were derived from 166 source proteins, including a wide range of intracellular and plasma proteins. These source proteins were substantially different than those identified previously from EBV cell lines.

All non-redundant HLA-DR-presented peptides identified from individuals were synthesized for testing with the same subjects' PBMC. The first subject peptides tested came from a youth with antibiotic-refractory Lyme arthritis (LA1) who had a synovectomy following persistent arthritis for one year, despite 9 months of oral and intravenous antibiotic therapy. He had one of the refractory arthritis-associate alleles: DRB1*0101. Of the 2,237 MS/MS spectra generated from his tissue sample, 464 had a consensus match identified with two or more mass spectrometry search programs (Mascot, OMSSA or X! Tandem), of which 104 were non-redundant. These 104 peptides, along with sixteen additional peptides identified by only one of three search programs, were synthesized and tested in a T-cell proliferation assay using the patient's own PBMC. Because of limited cell numbers, peptides were pooled (three per well) for testing.

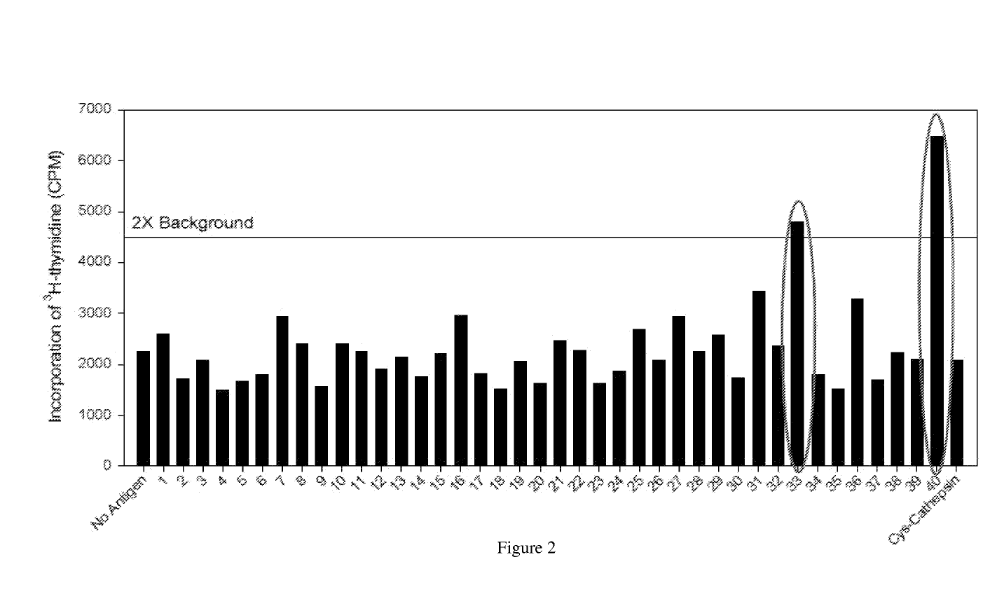

Only two peptide sets (set 33 and set 40) induced proliferation responses that were >2 times background (FIG. 2). Enough cells remained for retesting the first set together and the second peptide set individually. In the second assay, only one peptide induced proliferation and IFN-γ secretion that were >2 times background. This peptide (³⁴⁰LGRFERMLAAQGGVDPG³⁵⁵) (SEQ ID NO:1), 1 of the 16 identified by only one of the three search programs, originated from the source protein platelet-derived endothelial cell growth factor (ECGF), also called thymidine phosphorylase or gliostatin.

In vitro, ECGF acts as a chemotactic factor, causes a proliferative effect on endothelial cells, (Ishikawa et al., 338 Nature 557 (1989)); inhibits the growth of glial cells; and contributes to cortical neuron survival. Asai et al., 267 J. Biological Chem. 20311 (1992). In vivo, ECGF induces angiogenesis (Ishikawa et al., 1989); and is often over-expressed in many human cancers. Bronckaers et al., 29 Med. Res. Rev. 903 (2009). ECGF enzymatic activity involves the conversion of thymidine to thymine and deoxyribose-1-phosphate, and the thymidine-derived sugar is postulated to contribute to angiogenesis. Bijnsdorp et al., Biochemical Pharmacol. 786 (2010). Interestingly, most previously identified autoantigens in endocrine autoimmune diseases also have enzymatic activity. Aletha et al., 62 Arthritis Rheum 2569 (2010). ECGF, however, has not been identified previously as an autoantigen in any disease.

PBMC and serum samples from large numbers of patients with various manifestations of Lyme disease, from a rheumatoid arthritis comparison group, and from healthy control subjects, were tested for T- and B-cell reactivity with ECGF. All Lyme disease patients met the criteria of the Centers for Disease Control and Prevention for the diagnosis of this infection (39(RR-13) MMWR Morb. Mortl. Wkly Rpts. 1 (1990)), and those with RA met the ACR/EULAR criteria. Aletha et al., 2010. Bb was isolated from the skin lesions of all patients with EM tested here, and all patients with Lyme arthritis had high antibody responses to many Bb proteins by ELISA and immunoblotting. All control subjects were seronegative for Bb infection.

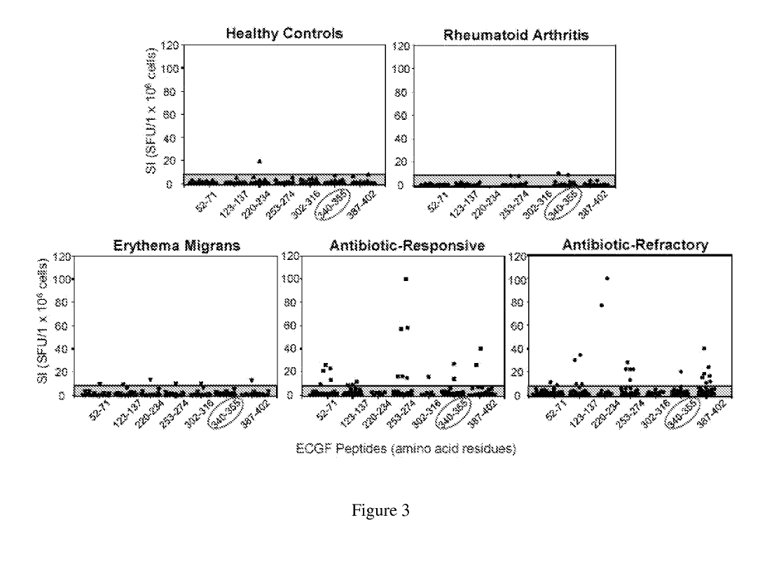

Initially, subjects' PBMC were tested using commercially available recombinant ECGF. ECGF inhibited thymidine incorporation into actively dividing cells (Takeuchi et al., 37 Arthritis Rheum. 662 (1994)), but it non-specifically induced PBMC to secrete IFN-γ. Consequently, three HLA-DR T-cell epitope prediction algorithms (Wang et al., PLoS Computat. Biol. e1000048 (2008)), were used to identify seven ECGF peptides, including the one peptide identified in the initial patients' sample (ECGF₃₄₀₋₃₆₅), that were predicted to be presented by the HLA-DR molecules associated with antibiotic-refractory Lyme arthritis. These peptides were synthesized and tested for autoreactivity via IFN-γ ELISPOT assays using patient and control PBMC (FIG. 3). Based upon the values obtained in 14 healthy control subjects (FIG. 3), a positive T cell response was defined as a stimulation index (no. of spots induced by an ECGF peptide /no. of spots in no antigen controls) 3 standard deviation (SD) above the mean of healthy controls, which characteristically gave >40 spot forming units (SFU)/10⁶ PBMC and a stimulation index of >8.

Only one of eighteen healthy control subjects (6%) had a low-response to a single ECGF peptide. In comparison, three of nineteen subjects with EM (16%) had responses which were slightly above background (P=0.6), eight of twenty-eight subjects with antibiotic-responsive arthritis (29%) had higher responses to ECGF peptides (P=0.07), and fourteen of thirty-eight subjects with antibiotic-refractory arthritis (36%) had similarly high responses to these peptides (P=0.02). Moreover, ten subjects with antibiotic-responsive or antibiotic-refractory arthritis had reactivity with two to four ECGF peptides, suggestive of epitope spreading. In comparison, only two of twelve patients with RA (17%) had reactivity with a single epitope that was only slightly above background. These results suggested that T cell autoreactivity to ECGF may be specific for Lyme disease; it may begin early in the infection, and it increases in frequency, magnitude and number of epitopes recognized later in the illness in patients with Lyme arthritis. Moreover, because only ˜20% of the ECGF protein sequence was tested in this analysis, the actual percentage of Lyme arthritis patients with ECGF T cell autoreactivity may be higher.

For an autoantigen to contribute to pathogenicity, it likely would need to induce both T- and B-cell responses. Therefore, IgG anti-ECGF antibodies were in patients' serum samples using two methods, ELISA and immunoblotting. A positive response by ELISA was defined as >3 SD above the mean value of healthy control subjects, and a positive immunoblot was defined by the presence of a band at the correct location for ECGF.

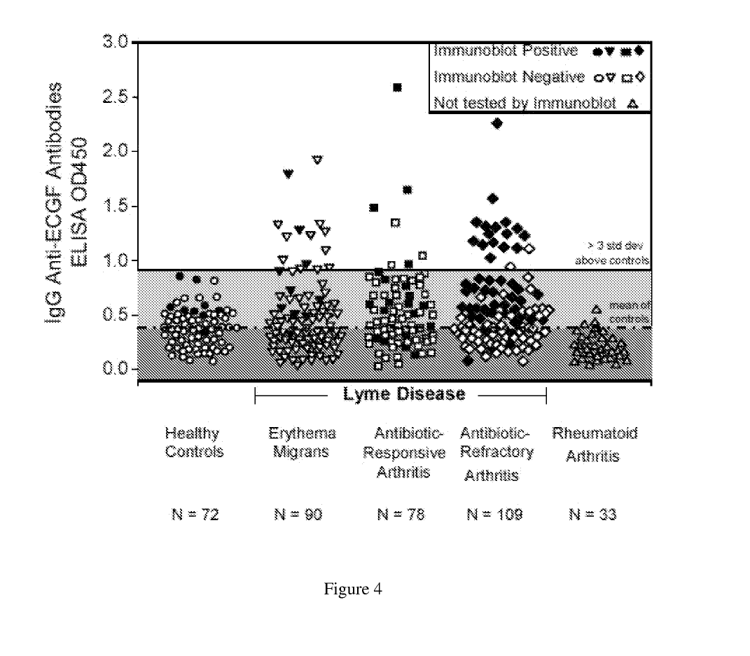

As determined by immunoblotting, serum samples from nine of seventy-two healthy control subjects (13%) had weak reactivity with ECGF (FIG. 4). In contrast, fifty of 109 patients (46%) with antibiotic-refractory arthritis had weak-to-strong responses to ECGF, which was significantly different than healthy subjects (P<0.001); twenty of seventy-eight patients (26%) with antibiotic-responsive arthritis and 10 of 90 patients (11%) with EM also had ECGF responses, but these frequencies were not significantly different than control subjects (P=0.07 and 1.0, respectively).

As determined by ELISA, none of seventy-two healthy control subject had a positive response compared with eighteen of 109 patients (17%) with refractory arthritis (P<0.001), seven of seventy-eight patients (9%) with responsive arthritis (P=0.03), fifteen of ninety EM patients (16%) (P<0.001), and none of thirty-three RA patients. Moreover, most of the patients in the refractory group who had an ELISA value above the mean value had a positive immunoblot, which was not the case in the other groups. Thus, autoantibodies to ECGF were sometimes present early in the infection or later in the illness in patients with antibiotic-responsive arthritis, but the frequency of these antibodies was significantly greater than healthy controls only in patients with antibiotic-refractory arthritis

For the autoantigen ECGF to have pathogenic relevance in antibiotic-refractory Lyme arthritis, one would predict that this protein would be present in high concentrations in patients' inflamed joints. Therefore, ECGF levels in joint fluid were determined by ELISA and its presence in synovial tissue identified using immunohistochemical techniques. Although joint fluid was available in patients with antibiotic-responsive arthritis, synovial tissue was not as therapeutic synovectomies are never necessary in this patient group.

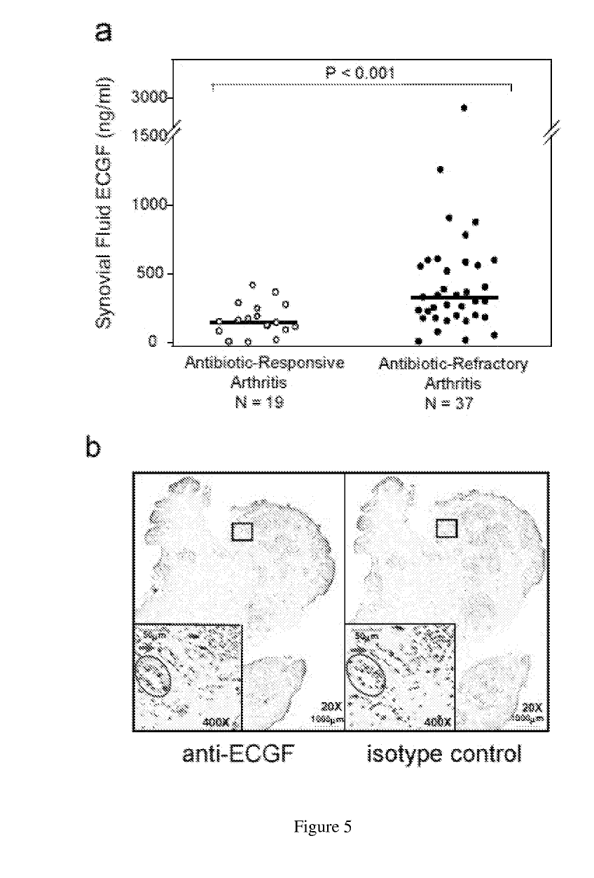

As determined by sandwich ELISA, subjects with antibiotic-refractory arthritis had very high concentrations of ECGF in synovial fluid (mean value=328 ng/ml±428), and these concentrations were significantly higher than those in subjects with antibiotic-responsive arthritis (142 ng/ml±119) (P<0.001) (FIG. 5a). Previously, other investigators showed that ECGF concentrations were also high in the joint fluids of RA patients (223 ng/ml±211, N=248), (Takeuchi et al., 37 Arthritis Rheum. 662 (1994)), similar to those levels found in patients with antibiotic-refractory Lyme arthritis. In contrast, osteoarthritis (OA) patients, a minimally inflammatory form of arthritis, had significantly lower ECGF levels (8.7 ng/ml±14.3) than subjects with either antibiotic-refractory arthritis or rheumatoid arthritis.

Synovial tissue samples from sixteen patients with antibiotic-refractory arthritis and five with RA were analyzed for the presence of ECGF. Of the sixteen patients with antibiotic-refractory arthritis, ten (63%) had moderate-to-intense staining for ECGF in the lining and sublining of the synovial tissue, four (25%) had mild staining, and two (12%) had no staining in these areas. A representative example of intense staining at these sites is shown in FIG. 5b. At high magnification (400×), ECGF staining was clearly evident in the sublining area around blood vessels (circle) and in large cells that were likely synovial fibroblasts (arrow). In contrast, three of the five patients with RA had only mild staining in lining areas, and two had only mild staining around blood vessels. Thus, in the majority of patients with antibiotic-refractory arthritis, large amounts of ECGF were present in joint fluid and synovial tissue where it could be presented by HLA-DR molecules on local antigen presenting cells.

The duration of arthritis prior to and after antibiotic therapy, HLA-DR alleles, and joint fluid cytokine and chemokine levels were compared in patients with antibiotic-refractory or antibiotic-responsive arthritis according to ECGF antibody responses, as shown in Table 1:

TABLE 1
Clinical findings: antibiotic-refractory or -responsive Lyme arthritis

| | Antibiotic- | Antibiotic- | |
|---|---|---|---|
| | refractory | responsive | P value |
| Total no. of patients | 109 | 78 | |
| Duration of arthritis | | | |
| (mos.), median (range) | | | |
| Onset to antibiotics | | | |
| Antibiotics to resolution | 11 (4-48) | 2 (1-3) | <0.001 |
| Antibody responses | | | |
| according to HLA-DR | | | |
| alleles | | | |
| No. of patients tested | 97 | 70 | |
| No. of alleles (%) | 194 | 140 | |
| 0101, 0102, 04* or 1501 | 99 (51%) | 53 (38%) | 0.02 |
| 0301, 0801, 1101, or 1104 | 36 (19%) | 42 (30%) | 0.02 |
| 0701 | 24 (12%) | 13 (9%) | 0.5 |
| Cytokines/chemokines** | | | |
| No. of patients tested | 37 | 19 | |
| Levels in joint fluid, pg/ml | | | |
| IFNγ | 13 | 3 | 0.01 |
| TNFα | 28 | 18 | 0.05 |
| IL-1β | 4 | 2 | 0.05 |
| IL-6 | 20,900 | 6,920 | 0.02 |
| IL-8 | 13,800 | 4,200 | 0.01 |
| CXCL9 | 354,000 | 119,200 | 0.03 |
| CXCL10 | 43,700 | 23,450 | <0.001 |
| CCL2 | 3,570 | 1,085 | <0.001 |
| CCL3 | 258 | 78 | 0.006 |
| CCL4 | 307 | 144 | 0.007 |

*The 04 alleles in this analysis were 0401, 0402, 0403, 0404, 0405, 0407, 0408, and 0409.
**Levels of the following cytokines and chemokines were also determined: CCL5, CXCL11, CXCL13, IL-7, IL-10, IL-12p40, IL-12p70, IL-17, IFNα, but the differences between the values in antibiotic refractory and responsive patients were not statistically significant.

Consistent with previous studies (Steer & Angelis, 54 Arthritis Rheum. 3079 (2006); Steere et al., 203 J. Experi. Med. 961 (2006); Shin et al., 56 Arthritis Rheum. 1325 (2007)), a number of significant differences were noted in these factors between patients with refractory or responsive arthritis but within each of these groups, significant differences were not found according to ECGF antibody responses. There was a trend, however, toward higher levels of CXCL9 in the sixteen patients who had positive ECGF antibody responses (determined by immunoblotting) compared with the twenty-four patients who lacked such responses (median value, 636,500 vs 313,000 pg/ml).

When the levels of 20 cytokines and chemokines in joint fluid were correlated with the joint fluid ECGF levels, a very strong direct correlation between the levels of IFN-γ and ECGF were found in patients with antibiotic-refractory arthritis (r=0.8, P=0.0000000001), as shown in Table 2:

TABLE 2
Correlation of ECGF protein levels with cytokines and chemokines concentrations in joint
fluid in patients with antibiotic-refractory and antibiotic-responsive Lyme arthritis

| | Antibiotic-refractory* | | | Antibiotic-responsive | | |
|---|---|---|---|---|---|---|
| | No. samples | | | No. samples | | |
| | w/detectable | Correlation | | w/detectable | Correlation | |
| | cytokine | Coefficient < | | cytokine | Coefficient < | |
| | levels¶ | value | P value | levels¶ | value | P value |
| Pro-inflammatory | | | | | | |
| cytokines | | | | | | |
| IFN-γ | 37 | 0.8 | 0.00000000001 | 18 | 0.1 | 0.6 |
| IFN-α | 26 | 0.6 | 0.003 | 15 | −0.07 | 0.8 |
| TNF | 36 | 0.7 | 0.00001 | 19 | 0.2 | 0.4 |
| IL-1β | 37 | 0.6 | 0.0002 | 18 | 0.03 | 0.9 |
| Macrophage | | | | | | |
| chemoattractants | | | | | | |
| CCL3 | 36 | 0.6 | 0.0002 | 16 | 0.09 | 0.7 |
| CCL4 | 36 | 0.5 | 0.0008 | 19 | 0.2 | 0.4 |
| Anti-inflammatory | | | | | | |
| cytokines | | | | | | |
| IL-10 | 36 | 0.5 | 0.0007 | 18 | 0.2 | 0.5 |
| IL-5 | 36 | 0.9 | 0.00000000004 | 12 | −0.1 | 0.7 |

*Only cytokines and chemokines are shown that correlated significantly with ECGF protein levels. Correlations were not found between ECGF values and the levels of CXCL9, CXCL10, CXCL11, CXCL13, IL-6, IL-7, IL-8, IL-12p40, IL-12p70, IL-17, IL-23, CCL2, and CCL5.
Total number of patients' joint fluid samples tested were 37 antibiotic-refractory and 19 antibiotic-responsive.

Similarly, there was a strong direct correlation in the refractory group between IL-5 and ECGF levels. In another system, a known autoantigen (thyroglobulin) induced patients' PBMC to secrete both IFNγ and IL-5, and IL-5 may enhance autoantibody production. Nielsen et al., 147 Clin. Exp. Immunol. 287 (2007). There was also a direct correlation in the refractory group between the levels of ECGF and several other inflammatory or anti-inflammatory cytokines and chemokines, but these correlations were not as strong as those with IFN-γ and IL-5. In contrast, there was no correlation between the levels of these inflammatory mediators and ECGF values in patients with antibiotic-responsive arthritis. The strong correlation between the levels of IFN-γ and ECGF in joint fluid suggests that IFN-γ might also have a role in inducing secretion of the autoantigen ECGF in the synovial lesion.

The present invention provides for a novel biomarker, ECGF, that is present in high concentrations in inflamed joints and induces T- and B-cell responses in some subjects with Lyme arthritis, particularly in those with antibiotic-refractory arthritis. ECGF might be considered a surprising autoantigen because its expression is not joint-specific. The K/B×N mouse model of autoimmune arthritis may also hold insights regarding this issue. In that model, joint-specific inflammation is due to immune recognition of the ubiquitous self-protein, glucose-6-isomerase (GPI), which is recognized by the single T-cell receptor in this transgenic mouse. Matsumoto et al., 3 Nat. Immunol. 360 (2002). GPI, which accumulates in high concentrations on articular surfaces, is bound by anti-GPI autoantibodies, which triggers the activation of complement and Fc receptors leading to autoimmune synovitis. Individual anti-GPI monoclonal antibodies do not induce arthritis, however; rather, anti-GPI antibodies recognizing multiple epitopes are required. Maccioni et al., 195 J. Exp. Med. 1971 (2002). Thus, although GPI is a ubiquitous self-antigen, pathology develops only in the localized environment of the joint where inflammatory responses to this protein are not regulated appropriately.

In comparison, human autoimmune disease is more complicated due to the diversity of immune responses. In RA, anti-cyclic citrullinated peptide (anti-CCP) antibodies, the first autoantibodies identified that are specific for this disease, are found in about 60% of RA patients (Lee & Schur, 62 Annals Rheum. Disease 870 (2003)), suggesting that other as yet to be identified autoantibodies may play a role in the disease. Moreover, anti-CCP antibodies may develop months or years before the onset of arthritis (Nielen et al., 50 Arthritis Rheum. 380 (2004)), suggesting that these antibodies may be necessary but are not sufficient to induce arthritis. Similarly, in antibiotic-refractory Lyme arthritis, antibody responses to ECGF were found significantly more often in patients with antibiotic-refractory arthritis, but not all refractory patients had anti-ECGF antibody responses. Furthermore, ECGF reactivity was sometimes found in patients with other early or late manifestations of the illness, but these patients did not have clinical autoimmune disease. Thus, as in RA, pathogenicity in antibiotic-refractory Lyme arthritis surely involves heterogenous, multifactorial processes; and other yet to be identified factors, including other specific autoantigens, may play a role in the pathogenesis of the disease.

There are a number of risk factors involved in antibiotic-refractory Lyme arthritis, including both spirochetal and host factors. For example, this disease course is triggered significantly more often by Bb RST1 strains, which account for 30%-50% of the infections in the northeastern U. S. Jones et al., 60 Arthritis Rheum. 2174 (2009). These strains are more inflammatory than other strains, including those found in Europe (Strle et al., 200 J. Infect. Dis. 1936 (2009)), particularly in individuals with a TLR1 polymorphism. Strle et al., Abstracts 12th Intl. Cong. Lyme Borreliosis Tick-Borne Dis. 12 (2010). Further, RST1-infected, antibiotic-refractory patients with this polymorphism had exceptionally high levels of IFNγ and the IFNγ-inducible chemoattractant CXCL9 in joint fluid. Shine et al., 56 Arthritis Rheum. 1325 (2007). Also, Bb, an extracellular pathogen, is known to bind certain host proteins (Hallstrom et al., 202 J. Infect. Dis. 490 (2010)), or tick proteins (Anguita et al., 19 Immun. 849 (2002)), to its surface to aid in its spread and survival. Perhaps ECGF binds directly to the surface of certain spirochetal strains resulting in simultaneous uptake and processing by antigen-presenting cells.

Alternately, T-cell epitope mimicry between a spirochetal and ECGF epitope presented by certain HLA-DR alleles might account for ECGF autoreactivity, although this seems less likely since no single ECGF epitope was recognized by all or even the majority of ECGF-reactive patients. Studies in an animal model of antibiotic-refractory Lyme arthritis suggest that certain HLA-DR alleles, such as DRB1*0401, lead to greater inflammatory responses. Iliopoulou et al., 60 Arthritis Rheum. 3831 (2009). Once established, the role of immune reactivity with ECGF may be to amplify joint inflammation, particularly since large amounts of this antigen are present in inflamed joints. Finally, an antibiotic-refractory course surely requires dysregulation of immune responses. It was shown previously that Th1 cells are abundant and enriched in synovial fluid in both antibiotic-refractory and antibiotic-responsive patients. In the refractory group, lower numbers of Treg correlated with slower resolution of arthritis. Shen et al., 62 Arthritis Rheum. 2127 (2010). High levels of IFNγ in joints, secretion of ECGF, upregulation of HLA-DR molecules, and high concentrations of the CD4+ T effector cell chemoattractant CXCL9 may help set the stage for the development of autoimmunity to ECGF.

The identification of ECGF for a biomarker in Lyme arthritis, as provided herein, is an important addition to the clinician's arsenal in combating chronic inflammatory arthritis, and assists the clinician in choosing the course of therapy. For example, when antibiotic-refractory Lyme arthritis is diagnosed, nonsteroidal anti-nflammatory drugs (NSAIDs) or intra-articular steroid; or disease modifying anti-rheumatic drugs (DMARDs), such as hydroxychloroquine or methotrexate, may be prescribed. Additionally or alternatively, anti-TNF therapy (e.g., HUMIRA® (adalimumab) or ENBREL® (etanercept)) may be beneficial. It is also possible that ECGF-specific therapy, such as targeting ECGF and/or ECGF-binding antibodies, may also be beneficial.

Importantly, the discovery-based methodology used herein to identify novel autoantigens is likely to have much broader applicability. The present invention shows that HLA-DR-presented peptides may be identified from synovectomy specimens of individual patients by tandem mass spectrometry, and immunogenic antigens may be uncovered in these patients by synthesis and testing of these peptides with the same patient's PBMC. This approach can be applicable to any form of chronic inflammatory arthritis, any autoimmune disease, or malignancy in which important immune responses are not yet known.

The present embodiments provide for a biomarker useful for the diagnosis of Lyme arthritis, in particular, antibiotic-refractory Lyme arthritis. More specifically, subject immunoreactivity (e.g., ECGF-reactive T-cells, and/or anti-ECGF antibodies) to ECGF, ECGF fragments or ECGF peptides or ECGF epitopes, such as, for example, LGRFERMLAAQGVDPG (SEQ ID NO:1), are indicative of Lyme arthritis in some, but not all, Lyme arthritis subjects. Additional ECGF peptides useful according to the present embodiments include, for example, ADIRGFVAAVVNGSAQGAQI (SEQ ID NO:2); DKVSLVLAPALAACG (SEQ ID NO:3); SKKLVEGLSALVVDV (SEQ ID NO:4); KTLVGVGASLGLRVAAALTAMD (SEQ ID NO:5); LRDLVTTLGGALLWL (SEQ ID NO:6); and GTVELVRALPLALVLH (SEQ ID NO:7).

For example, an immunoassay can be used to identify antibodies present in a serum sample that bind ECGF. The immunoassay can be an ELISA, agglutination test, direct immunofluorescence assay, indirect immunofluorescence assay, western blot, an immunoblot assay, and the like. For example, the immunoassay could be an immunoblot that carries a recombinant ECGF or ECGF peptide(s). The immunoassay can also include or be used with an immunoassay (e.g., a kit) that include Borrelia antigens known in the art, such as p83/100 derived from strain PKo (Borrelia afzelii); p39 (BmpA) and OspC from strains PKa2 (B. burgdorferi sensu stricto), PBi (B. garinii, OspA-type 4), and PKo; p4 μl (internal flagellin fragment) from PKo and PBi; p58 derived from PBi; Osp17 from PKo; decorin binding protein A (DbpA) derived from B. garinii strain PBr (OspA-type 3); V1sE from B. burgdorferi sensu stricto strain PKa2; and/or OspC from B. garinii strain 20047. See, e.g., 41 J. Clin. Microbiol. 1299 (2003); Wilske et al., 188 Med. Micriobiol. Immunol. 139 (1999). It should be noted that the Borrelia antigen(s) can be derived from natural and/or recombinant sources. For example, the present ECGF autoantigen can be included in or used in conjunction with an immunoassay such as the BORRELIA VIRASTRIPE® IgG, IgM test kit (Viramed Biotech AG, Planegg, Germany). The BORRELIA VIRASTRIPE® is an immunoblot that carries native, purified antigens from Borrelia afzelii (Pko), Borrelia burgdorferi sensu stricto, and recombinant Borrelia antigen V1sE. Alternatively, the present ECGF autoantigen may be included in or used in conjunction with an immunoassay such as the BORRELIA B31 VIRABLOT® Western blot test kits (Viramed Biotech AG, Planegg, Germany) which identify anti-Borrelia antigen-binding IgG and/or IgM in the serum of suspected Borrelia-infected patients. For immunoassays designed to identify ECGF autoantigen-binding antibodies in the serum of inflammatory arthritis subjects, the serum sample can be pre-enriched for antibodies by methods known in the art.

Further, ECGF-reactive T-cells in PBMC and SFMC can be assessed using a number of assays. For example, ECGF-reactive T-cells in PBMC and SFMC can be assessed using tetramer reagents comprising recombinant HLA-DR molecules and ECGF epitopes. “Epitope” refers to that portion of any molecule capable of being recognized by, and bound by, an antibody (the corresponding antibody binding region may be referred to as a paratope), and/or eliciting an immune response. In general, epitopes consist of chemically active surface groupings of molecules, e.g., amino acids, and have specific three-dimensional structural characteristics as well as specific charge characteristics.

The ECGF peptides may comprise naturally occurring or analog or derivative amino acids, as long as the immunoreactive or immunostimulatory nature of the peptide is retained to sufficient degree to allow T-cell activation and/or antibody binding. Thus, some amino acids may be added to or subtracted from the native ECGF or ECGF peptides as known in the art. Additionally, some amino acids of the native human ECGF or ECGF peptides may be substituted with amino acids that occur in other species, or be substituted as known in the art. Amino acid substitution exchange groups and empirical similarities between amino acid residues, can be found in standard texts such as Schulz et al., PRINCIPLES OF PROTEIN STRUCTURE, 14-16 (Springer-Verlag, New York, 1979). There is a limit to how much substitution can be tolerated before the original tertiary structure is lost. Typically, tertiary structure conservation would be lost when the amino acid sequence varies by more than 50%. See, e.g., Chothia & Lesk, Relation between the divergence of sequence & structure in proteins, 5 EMBO J. 823 (1986). Guidance concerning which amino acid changes are likely to be phenotypically silent is found in Bowie et al., 247 Science 1306 (1990). Amino acids that are essential for function can be identified by methods known in the art, such as site-directed mutagenesis or alanine-scanning mutagenesis. Cunningham et al., 244 Science 1081 (1989). The latter procedure introduces single alanine mutations at every residue in the molecule. The resulting mutant molecules are then tested for biological activity such as antibody binding and/or T-cell stimulation. Sites that are critical for ligand-receptor binding can also be determined by structural analysis such as crystallography, nuclear magnetic resonance, or photoaffinity labeling. Smith et al., 224 J. Mol. Biol. 899 (1992); de Vos et al., 255 Science 306 (1992).

The gene for human ECGF has been sequenced and is available at numerous sources such as the NCBI web site, GeneID: 1890 (UniProtKB/Swiss-Prot: P19971). Further, the ECGF gene is conserved in human, chimpanzee, rat, and zebrafish. Hence, ECGF peptides can include those derived from non-human sources or appropriate sequence information. In an aspect of the invention, the ECGF autoantigen is predicted to be presented by HLA-DR molecules associated with chronic inflammatory arthritis.

As noted above, generally, amino acid substitutions should be made conservatively; i.e., a substitute amino acid should replace an amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Variants within the scope of this invention may also, or alternatively, contain other modifications, including the deletion or addition of amino acids, that have minimal influence on the stimulatory properties, antibody binding, tertiary structure of the peptide. Thus, for example, in the amino acid sequence LGRFERMLAAQGVDPG (SEQ ID NO:1), conservative substitutions allow for an amino acid sequence X³X³X⁴X⁵X¹X⁴X³X³X¹X¹X¹X¹X³X¹X¹, wherein X¹ is ala, pro, gly, glu, asp, gln, asn, ser, thr; X³ is val, ile, leu, met, ala, phe; and X⁴ is lys, arg, his; X⁵ is phe, tyr, trp, his; with the proviso that functional activity is retained to a meaningful degree such that the particular assay (e.g., ECGF T-cell reactivity or immunoassay) works as intended to provide evidence in diagnosing subjects with Lyme arthritis.

Moreover, peptides often contain amino acids other than the twenty “naturally occurring” amino acids. Further, many amino acids, including the terminal amino acids, may be modified by natural processes, such as processing and other post-translational modifications, or by chemical modification techniques well known in the art. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Creighton, PROTEINS—STRUCTURE & MOLECULAR PROPERTIES (2nd ed., W.H. Freeman & Co., New York, 1993). Many detailed reviews are available on this subject, such as by Wold, POSTTRANSLATIONAL COVALENT MODIFICATION OF PROTEINS, 1-12 (Johnson, ed., Academic Press, New York, 1983); Seifter et al. 182 Meth. Enzymol. 626 (1990); Rattan et al., 663 Ann. N.Y. Acad. Sci. 48 (1992). Accordingly, the peptides of the present invention also encompass derivatives or analogs in which a substituted amino acid residue is not one encoded by the genetic code.

Further, “derivatives” of an ECGF, ECGF peptide or ECGF epitope contain additional chemical moieties not normally a part of the protein. Covalent modifications of ECGF or ECGF peptides/epitopes are included within the scope of this invention. Such modifications may be introduced into the molecule by reacting targeted amino acid residues of the antibody with an organic derivatizing agent that is capable of reacting with selected side chains or terminal residues. For example, derivatization with bifunctional agents, well-known in the art, is useful for cross-linking the antibody or fragment to a water-insoluble support matrix or to other macromolecular carriers. Derivatives also include radioactively labeled peptides that are labeled, for example, with radioactive iodine (¹²⁵I, ¹³¹I), carbon (¹⁴C), sulfur (³⁵S), tritium (³H) or the like; conjugates of peptides with biotin or avidin, with enzymes, such as horseradish peroxidase, alkaline phosphatase, β-D-galactosidase, glucose oxidase, glucoamylase, carboxylic acid anhydrase, acetylcholine esterase, lysozyme, malate dehydrogenase or glucose 6-phosphate dehydrogenase; and also conjugates of monoclonal antibodies with bioluminescent agents (such as luciferase), chemoluminescent agents (such as acridine esters), or fluorescent agents (such as phycobiliproteins).

Structural analogs of ECGF and ECGF peptides/eptiopes of the present invention are provided by known method steps based on the teaching and guidance presented herein. Knowledge of the three-dimensional structures of proteins is crucial in understanding how they function. The three-dimensional structures of hundreds of proteins are currently available in protein structure databases (in contrast to the thousands of known protein sequences in sequence databases). Analysis of these structures shows that they fall into recognizable classes of motifs. It is thus possible to model a three-dimensional structure of a protein based on the protein's homology to a related protein of known structure. Many examples are known where two proteins that have relatively low sequence homology, can have very similar three dimensional structures or motifs.

It is possible to determine the three dimensional structures of proteins of up to about 15 kDa by nuclear magnetic resonance (NMR). The technique only requires a concentrated solution of pure protein. No crystals or isomorphous derivatives are needed. The structures of a number of proteins have been determined by this method. The details of NMR structure determination are well-known in the art. See, e.g., Wuthrich, NMR of Proteins & Nucleic Acids (Wiley, N.Y., 1986); Wuthrich, 243 Science 45 (1989); Clore et al., 24 Crit. Rev. Bioch. Molec. Biol. 479 (1989); Cooke et al., 8 Bioassays 52 (1988).

Thus, according to the present invention, use of NMR spectroscopic data can combined with computer modeling to arrive at structural analogs of at least portions of ECGF, ECGF peptides and/or ECGF epitopes based on a structural understanding of the topography. Using this information, one of ordinary skill in the art can achieve structural analogs of ECGF, ECGF peptides and/or ECGF epitopes such as by rationally-based amino acid substitutions allowing the production of peptides in which the ECGF binding affinity or avidity is modulated in accordance with the requirements of the expected diagnostic use of the molecule, for example, the achievement of greater binding specificity or affinity.

Thus, one embodiment of the present invention provides for a kit for identifying a subject with chronic inflammatory arthritis, particularly Lyme arthritis, comprising ECGF or a set of synthesized ECGF peptides or ECGF epitopes, and reagents necessary for conducting an immunoassay, wherein the immunoassay is capable of detecting the presence of an antibody in a sample obtained from said subject that binds to ECGF or a portion or fragment thereof, such as a set of synthesized ECGF peptides/epitopes.

ECGF is available commercially, for example from R&D Systems, Inc. (Minneapolis, Minn.). Additionally, ECGF can be obtained from platelets, liver, lung, placenta, spleen, lymph nodes, peripheral lymphocytes, and astrocytes. See Haraguchi et al., 368 Nature 198 (1994); Toi et al., 6 Lancet Oncol. 158 (2005); Akiyama et al., 95 Canc. Sci. 851 (2004).

In a particular embodiment, the ECGF peptides/epitopes are one or more of the peptides having the following amino acids: LGRFERMLAAQGVDPG (SEQ ID NO:1); ADIRGFVAAVVNGSAQGAQI (SEQ ID NO:2); DKVSLVLAPALAACG (SEQ ID NO:3); SKKLVEGLSALVVDV (SEQ ID NO:4); KTLVGVGASLGLRVAAALTAMD (SEQ ID NO:5); LRDLVTTLGGALLWL (SEQ ID NO:6); GTVELVRALPLALVLH (SEQ ID NO:7); or functionally equivalent portions, fragments, analogs, or derivatives of any of these. The immunoassay in the kit may be an enzyme-linked immunosorbent assay (ELISA) or immunoblot, the components for which are well-known in the art. The sample obtained from the subject may be peripheral blood, serum, synovial fluid, synovial tissue, peripheral blood mononuclear cells (PBMC), or the synovial fluid mononuclear cells (SFMC). The subject may be a mammal, such as a human. The peptides may be synthesized or obtained from natural or recombinant sources, each of which is well-known in the art.

Another embodiment provides for a method for determining whether a biological sample, (for example, a sample comprising PBMC or SFMC), bears T-cells reactive to ECGF or ECGF peptides or epitopes by providing a set of at least one synthesized ECGF peptide or epitope that are predicted to be presented by HLA-DR molecules; stimulating the biological sample with at least one of the ECGF peptides/epitopes (or its functionally equivalent portion, analog, or derivative); and measuring T-cell proliferation in vitro or secretion of IFN-γ into cell culture supernatants as a test for T-cell reactivity.

Another embodiment provides for a method for determining whether a subject, suffering from chronic inflammatory arthritis (for example Lyme arthritis, and more particularly antibiotic-refractory Lyme arthritis), bears T-cells reactive to ECGF or ECGF peptides or epitopes by providing a set of at least one synthesized ECGF Peptide or Epitope (or its functionally equivalent portion, analog, or derivative) that are predicted to be presented by HLA-DR molecules; stimulating PBMC or the SFMC obtained from the subject with at least one of the ECGF peptides or epitopes; and measuring T-Cell proliferation in vitro or secretion of IFN-γ as a test for T-cell reactivity. The term “subject” as used herein refers to any individual or patient to which the subject methods are performed. Generally the subject is human, although as will be appreciated by those in the art, the subject may be an animal. Thus, other animals, including mammals such as rodents (including mice, rats, hamsters and guinea pigs), cats, dogs, rabbits, farm animals including cows, horses, goats, sheep, pigs, etc., and non human primates (including monkeys, chimpanzees, orangutans and gorillas) are included within the definition of subject.

Another embodiment provides for a method for determining whether a subject, suffering from chronic inflammatory arthritis, has a B cell response to ECGF as determined by the detection of anti-ECGF antibodies in serum, comprising the steps of: (a) providing an isolated autoantigen, wherein said autoantigen is ECGF, ECFG peptide, ECGF epitope, or a functionally equivalent portion, fragment, analog or derivative thereof; (b) providing a biological sample from a subject; (c) conducting an immunoassay on said sample utilizing said autoantigen; wherein said immunoassay detects the presence of antibodies that recognize said ECGF; and (d) determining that the subject contains an antibody against said autoantigen if the results of the immunoassay indicate that an antibody that recognizes said autoantigen is present in said sample. For example, the subject suffers from antibiotic-refractory Lyme arthritis or antibiotic-responsive Lyme arthritis. The immunoassay can be an immunoblot or an ELISA, etc. The immunoassay may also include Borrelia antigens. The immunoassay may also include other antigens associated with arthritis.

The identification of ECGF autoimmune response in a subject with inflammatory arthritis aids the clinician in diagnosing and/or treating the arthritis indication. For example, presence of ECGF autoantibody (e.g., identified by immunoblot) indicates that autoimmune disease is involved in the subject's inflammation.

The present invention can be defined in any of the following numbered paragraphs:

1. A method for determining whether a biological sample obtained from a subject is reactive with endothelial cell growth factor (ECGF) autoantigen comprising contacting said biological sample with an immunoassay comprising at least one ECGF autoantigen and/or autoantigenic epitope.

2. The method of paragraph 1, wherein the immunoassay identifies the presence of antibodies that bind an ECGF autoantigen.

3. The method of paragraph 1, wherein said immunoassay is an ELISA, agglutination test, direct immunofluorescence assay, indirect immunofluorescence assay, or an immunoblot assay.

4. The method of paragraph 1, wherein the immunoassay is a T-cell proliferation assay.

5. The method of paragraph 4, wherein the T-cell proliferation assay is a ³H-thymdine incorporation assay, CFSE dilution, or an ELISPOT.

6. The method of paragraph 1, wherein the immunoassay is a T-cell reactivity assay.

7. The method of paragraph 6, wherein the immunoassay comprises measuring secretion of IFN-γ or other cytokines and/or chemokines.

8. The method of any one of the preceding paragraphs, wherein said ECGF autoantigen is ECGF, ECGF protein fragment, or at least one ECGF peptide selected from the group consisting of:

(SEQ ID NO: 1)
LGRFERMLAAQGVDPG;
(SEQ ID NO: 2)
ADIRGFVAAVVNGSAQGAQI;
(SEQ ID NO: 3)
DKVSLVLAPALAACG;
(SEQ ID NO: 4)
SKKLVEGLSALVVDV;
(SEQ ID NO: 5)
KTLVGVGASLGLRVAAALTAMD;
(SEQ ID NO: 6)
LRDLVTTLGGALLWL;
(SEQ ID NO: 7)
GTVELVRALPLALVLH;

or functionally equivalent analog or derivative of thereof.

9. The method of any one of the preceding paragraphs, wherein said biological sample is obtained from peripheral blood, synovial fluid, synovial tissue, peripheral blood mononuclear cells (PBMC), or synovial fluid mononuclear cells (SFMC).

10. The method of any one of the preceding paragraphs, wherein a positive result of immunoreactivity of the biological sample with the ECGF autoantigen is indicative of Lyme arthritis.

11. The method of paragraph 10, further comprising the step of treating the subject with a non-steroidal anti-inflammatory or disease modifying anti-rheumatic drugs.

12. The use of an isolated ECGF autoantigen as a biomarker for diagnosing Lyme arthritis.

13. The isolated ECGF autoantigen of paragraph 12, wherein said ECGF autoantigen is selected from the group consisting of:

ECGF;
(SEQ ID NO: 1)
LGRFERMLAAQGVDPG;
(SEQ ID NO: 2)
ADIRGFVAAVVNGSAQGAQI;
(SEQ ID NO: 3)
DKVSLVLAPALAACG;
(SEQ ID NO: 4)
SKKLVEGLSALVVDV;
(SEQ ID NO: 5)
KTLVGVGASLGLRVAAALTAMD;
(SEQ ID NO: 6)
LRDLVTTLGGALLWL;
(SEQ ID NO: 7)
GTVELVRALPLALVLH;

or functionally equivalent analog or derivative of thereof.

14. A method for determining whether a subject, suffering from chronic inflammatory arthritis, bears T-cells reactive to endothelial cell growth factor (ECGF) or ECGF peptides or ECGF epitopes comprising the steps of: (a) providing a set of synthesized ECGF Peptides or Epitopes that are predicted to be presented by HLA-DR molecules associated with chronic inflammatory arthritis; (b) stimulating peripheral blood mononuclear cells (PBMC) or the synovial fluid mononuclear cells (SFMC) with one of said ECGF Peptides or Epitopes; and (c) measuring T-Cell proliferation in vitro or secretion of IFN-γ into cell culture supernatants as a test for T-cell reactivity.

15. The method of paragraph 14, wherein the subject suffers from antibiotic-refractive Lyme arthritis.

16. The method of paragraph 15, wherein the subject suffers from antibiotic-responsive Lyme arthritis.

17. A method for determining whether a subject, suffering from chronic inflammatory arthritis, contains a B-cell response to ECGF resulting in the production of autoantibodies found in serum or synovial fluid against ECGF, comprising the steps of: (a) providing an isolated antigen, wherein said antigen is ECGF, ECGF peptide or ECGF epitope or a functionally equivalent portion, fragment, analog or derivative thereof; (b) providing a biological sample from a subject; (c) conducting an immunoassay on said sample utilizing said antigen; wherein said immunoassay detects the presence of antibodies that recognize said ECGF; and (d) determining that the subject contains an antibody against said antigen if the results of the immunoassay indicate that an antibody that recognizes said antigen is present in said sample.

18. The method of paragraph 17, wherein the subject suffers from antibiotic-refractory Lyme arthritis.

19. The method of paragraph 18, wherein the subject suffers from antibiotic-responsive Lyme arthritis.

20. The method of paragraph 18, wherein the immunoassay is an enzyme-linked immunosorbent assay (ELISA).

21. A kit for identifying a patient with chronic inflammatory arthritis comprising: ECGF or a set of synthesized ECGF peptides, and reagents necessary for conducting an immunoassay, wherein the immunoassay is capable of detecting the presence of an antibody in a sample, and wherein the antibody is capable of binding to said antigen.

22. The kit of paragraph 21, wherein the chronic inflammatory arthritis is antibiotic-resistant Lyme arthritis.

23. The kit of paragraph 21, wherein the chronic inflammatory arthritis is antibiotic-responsive Lyme arthritis.

24. The kit of paragraph 21, wherein the immunoassay is an enzyme-linked immunosorbent assay (ELISA).

25. The kit of paragraph 21, wherein the immunoassay is a western blot.

26. The kit of any one of paragraphs 21-25, wherein said kit further comprises Borrelia antigens.

### EXAMPLES

### Example 1

### ELISPOT Assays and Synthetic Peptides

Enzyme-linked immunosorbent spot (ELISPOT) assays were performed using ELISpot^{plus} for human IFN-γ kits (Mabtech Inc., #3420-2AW-Plus). Briefly, PBMC collected using Ficoll-Hypaque density centrifugation and stored in liquid nitrogen were thawed quickly and plated in round bottom, 96-well plates (Costar, #3799) at 2×10⁵ per well in 200 μl of complete media (RPMI-1640, 2 mM glutamine, 100 units/ml penicillin 100 μg/ml streptomycin, 10 mM HEPES (all from Invitrogen) and 10% human AB serum (Cellgrow). Peptides were added at a concentration of 1 μM in duplicate wells. Positive and negative controls consisted of 1% PHA (Invitrogen, #10576-015) and no antigen, respectively. After 5 days at 37° C. and 5% CO₂, cells were transferred to ELISPOT plates (Mabtech), previously coated with IFN-γ capture antibody, and incubated overnight. All subsequent steps were performed as detailed in the manufacturer's protocol. Images of wells were captured using ImmunoSpot series 3B analyzer and spots counted using ImmunoSpot 5.0 academic software (Cellular Technology Limited).

The human ECGF has the amino acid sequence:

| | |
|---|---|
| 1 | maalmtpgtg appapgdfsg egsqglpdps pepkqlpeli rmkrdggrls eadirgfvaa |
| 61 | vvngsaqgaq igamlmairl rgmdleetsv ltqalaqsgq qlewpeawrq qlvdkhstgg |
| 121 | vgdkvslvla palaacgckv pmisgrglgh tggtldkles ipgfnviqsp eqmqvlldqa |
| 181 | gccivgqseq lvpadgilya ardvtatvds lplitasils kklveglsal vvdvkfggaa |
| 241 | vfpnqeqare laktlvgvga slglrvaaal tamdkplgrc vghaleveea llcmdgagpp |
| 301 | dlrdlvttlg gallwlsgha gtqaqgaarv aaalddgsal grfermlaaq gvdpglaral |
| 361 | csgspaerrq llprareqee llapadgtve lvralplalv lhelgagrsr ageplrlgvg |
| 421 | aellvdvgqr lrrgtpwlrv hrdgpalsgp qsralqealv lsdrapfaap spfaelvlpp |
| 481 | qq |

All peptides were synthesized and HPLC purified at Tufts University Core Facility (Boston, Mass.). The ECGF peptides sequences were as follows:

(SEQ ID NO: 2)
ECGF₅₂₋₇₁ ADIRGFVAAVVNGSAQGAQI;
(SEQ ID NO: 3)
ECGF₁₂₃₋₁₃₇ DKVSLVLAPALAACG;
(SEQ ID NO: 4)
ECGF₂₂₀₋₂₃₄ SKKLVEGLSALVVDV;
(SEQ ID NO: 5)
ECGF₂₅₃₋₂₇₄ KTLVGVGASLGLRVAAALTAMD;
(SEQ ID NO: 6)
ECGF₃₀₂₋₃₁₆ LRDLVTTLGGALLWL;
(SEQ ID NO: 1)
ECGF₃₄₀₋₃₅₅ LGRFERMLAAQGVDPG (index case);
and
(SEQ ID NO: 7)
ECGF₃₈₇₋₄₀₁ GTVELVRALPLALVLH.

### Example 2

### ELISA Assays

Serum Anti-ECGF Antibody ELISA:

EasyWash ELISA plates (CoStar) were coated with 100 μl of 0.5 μg/ml carrier free, recombinant human PD-ECGF (R&D Systems, #229-PE/CF) dissolved in PBS and incubated overnight at 4° C. All subsequent steps were performed at room temperature with plates on a platform shaker set at 200 rpm. The next day, plates were washed three times with PBST (phosphate buffered saline and 0.05% Tween-20) then incubated with 200 μl of blocking buffer (5% nonfat dry milk in PBST) for 1 hr. Afterwards, wells were washed three times with PBST and 100 μl of each patient's serum sample diluted 1/100 with blocking buffer was added to individual wells and incubated for 1 hr. As a control, serum from eight healthy subjects was added to each plate to be used for inter-plate standardization. After three more washes with PBST, 100 μl goat anti-human IgG conjugated to horseradish peroxidase (KPL #074-1006) diluted 1:7500 in blocking buffer was added to each well and incubated for 1 hr. Plates were then washed three times with PBST, followed by three times with PBS and incubated with 100 μl of a 1:1 mixture of the substrate 3,3′,5,5′-tetramethylbenzidine and 0.01% hydrogen peroxide (TMB substrate reagent kit, #555214) (BD Biosciences). The reaction was stopped after 3 min with 100 μl of 2N sulfuric acid (LabChem. Inc., #LC25790-2). Absorbance values (OD₄₅₀) for each well were determined using a microplate reader (Bio-Rad, model 550).

Synovial Fluid ECGF Sandwich ELISA:

EasyWash ELISA plates were coated with 50 μl of the capture antibody, goat anti-human PD-ECGF (Santa Cruz, # SC-9523) diluted in PBS (5 μg/ml) and incubated overnight at 4° C. All subsequent steps were performed at room temperature. The next day, plates were washed three times with PBS and incubated with blocking buffer for 30 min. Afterwards, plates were washed three times with PBS and 100 μl of each patients' synovial fluid sample diluted 1:10 with blocking buffer was added to individual wells and incubated for 2 hr. In order to quantify results, recombinant human PD-ECGF serially diluted with blocking buffer were also added to each plate to generate a standard curve. After washing the plates three times, wells were filled with 150 μl of blocking buffer, gently vortexed and washed again three times with PBS to ensure removal of all unbound proteins. Plates were then incubated with 50 μl of the mouse anti-human PD-ECGF antibody (Santa Cruz, SC-47702) diluted in blocking buffer (5 ng/ml) for 2 hr. Plates were again washed with PBS and 50 μl of the detection antibody, goat anti-mouse IgG conjugated to horse radish peroxidase (Santa Cruz, #SC-2005) diluted in blocking buffer (1:1000) was added to plates and incubated for 1 hr. After plates were washed three times with PBS, 100 μl of TMB was added for ˜6 min and then the reaction was stopped with 100 μl of 2N sulfuric acid. Plates were read as described above.

### Example 3

### Immunoblotting

Human recombinant PD-ECGF (12 μg) was electrophoresed through a 10% mini-PROTEAN TGX gels (Bio-Rad) then transferred to nitrocellulose membranes. All subsequent steps were performed at room temperature with rocking. Membranes were cut into strips, individually placed into eight channel reservoir liners (Costar, #4878) and incubated for 1 hr in 1.5 ml blocking buffer (5% nonfat dry milk, 0.1% Tween-20 in 20 mM Tris, 500 mM sodium chloride; pH 7.5). Afterwards, strips were washed three times for 1 min intervals with rinse buffer (0.1% Tween-20 in 20 mM Tris, 500 mM sodium chloride; pH 7.5) and each individual strip was incubated for 1 hr with patient's serum diluted 1:100 in blocking buffer. Strips were again washed three times with rinse buffer and incubated for 1 hr with goat anti-human IgG antibody conjugated to alkaline phosphatase (KPL, #4751-1006) diluted 1:2000 in blocking buffer. Strips were washed three times with rinse buffer and another three times with 20 mM Tris, 500 mM sodium chloride; pH 7.5. Bands were visualized by incubation with NBT/BCIP substrate solution (Roche Diagnostics GmbH, #11681451001) for 3-5 min after which the strips were washed with copious amounts of water to stop the reaction. Bands were considered positive if darker than the pre-determined positive control sample included in each assay.

### Example 4

### Immunohistochemical Characterization

Synovial tissue biopsies obtained from antibiotic-refractory Lyme arthritis patients after synovectomies were placed in optimal cutting temperature (Tissue-Tek, Sakura, Japan) and stored in liquid nitrogen. Subsequently, 6-8 μm-thick cryosections were cut and stored at −70° C. until use. After an initial review by hematoxylin-eosin of the biopsies; sections selected were those in which lining, sublining and subsynovium were present.

The presence of ECGF was assay by immunohistochemital staining performing the immunoperoxidase technique. The sections were fixed in cold acetone for 3 min and air dried. Fixed sections were washed in PBS. Endogenous peroxidase was blocked by incubating the sections with 3% hydrogen peroxide in methanol for 10 min. After rinsing with PBS three times, nonspecific reaction was blocked by incubating sections in 1× power block solution (Biogenex cat. No. HK085-5K) containing 10% normal donkey serum. The sections were then incubated at 4° C. overnight with appropriate dilution (3 μg/ml) of anti-rabbit polyclonal PD-ECGF (Abcam Cat. No. ab75920). Negative controls were done using nonspecific rabbit IgG (Sigma) as the primary antibody at the same IgG concentrations. After 5 min rinses with PBS, the sections were incubated with biotinylated anti-rabbit secondary antibody (Biogenex Cat. No. HK3260709) for 40 min at room temperature, rinsed in PBS, and incubated with peroxidase-streptavidin (Biogenex HK320-UK) for 20 min. After three rinses with PBS, the sections were incubated with diamiobenzidine substrate (Biogenex HK130-5K) for up to 10 min. The sections were washed in distilled water and counterstained with Mayer's hemotoxylin, and glycerol-mounted. Microscopic images were obtained with a Nikon eclipse ME6000 microscope using a Nikon digital camera DXM1200C. The intensity of PD-ECGF staining on each synovial tissue regions (lining, sublining and sub synovium) was graded on an arbitrary scale of 0-3: 0=no ECGF positive stain cells; 1=few (−50) positive staining cells; 2=many (−50 to 100) positive staining cells; and 3=most (>100) positive cells determined by counting a total of five 200× microscopic fields on for each synovial tissue regions (lining, sublining, and subsynovium); to sample a larger area per section every fifth 200× microscopic field was examined. For illustration purposes, whole-slide imaging of five random biopsies with moderate to intense stain in all synovial regions were scanned using with Mirax Viewer Scan (Carl Zeiss), at 20×/0.8 Plan-Apochromat objective and image was prepared using Mirax viewer software, as shown in FIG. 5.

1. A method for determining whether a biological sample obtained from a subject is reactive with endothelial cell growth factor (ECGF) autoantigen comprising contacting said biological sample with an immunoassay comprising at least one ECGF autoantigen and/or autoantigenic epitope.

2. The method of claim 1, wherein the immunoassay identifies the presence of antibodies that bind an ECGF autoantigen.

3. The method of claim 1, wherein said immunoassay is an ELISA, agglutination test, direct immunofluorescence assay, indirect immunofluorescence assay, or an immunoblot assay.

4. The method of claim 1, wherein the immunoassay is a T-cell proliferation assay.

5. The method of claim 4, wherein the T-cell proliferation assay is a ³H-thymdine incorporation assay, CFSE dilution, or an ELISPOT.

6. The method of claim 1, wherein the immunoassay is a T-cell reactivity assay.

7. The method of claim 6, wherein the immunoassay comprises measuring secretion of IFN-γ or other cytokines and/or chemokines.

8. The method of claim 1, wherein said ECGF autoantigen is ECGF, ECGF protein fragment, or at least one ECGF peptide selected from the group consisting of:
(SEQ ID NO: 1)
LGRFERMLAAQGVDPG;
(SEQ ID NO: 2)
ADIRGFVAAVVNGSAQGAQI;
(SEQ ID NO: 3)
DKVSLVLAPALAACG;
(SEQ ID NO: 4)
SKKLVEGLSALVVDV;
(SEQ ID NO: 5)
KTLVGVGASLGLRVAAALTAMD;
(SEQ ID NO: 6)
LRDLVTTLGGALLWL;
(SEQ ID NO: 7)
GTVELVRALPLALVLH;
or functionally equivalent analog or derivative of thereof.

9. The method of claim 1, wherein said biological sample is obtained from peripheral blood, synovial fluid, synovial tissue, peripheral blood mononuclear cells (PBMC), or synovial fluid mononuclear cells (SFMC).

10. The method of claim 1, wherein a positive result of immunoreactivity of the biological sample with the ECGF autoantigen is indicative of Lyme arthritis.

11. The method of claim 10, further comprising the step of treating the subject with a non-steroidal anti-inflammatory or disease modifying anti-rheumatic drugs.

12. (canceled)

13. (canceled)

14. A method for determining whether a subject, suffering from chronic inflammatory arthritis, bears T-cells reactive to endothelial cell growth factor (ECGF) or ECGF peptides or ECGF epitopes comprising the steps of: (a) providing a set of synthesized ECGF Peptides or Epitopes that are predicted to be presented by HLA-DR molecules associated with chronic inflammatory arthritis; (b) stimulating peripheral blood mononuclear cells (PBMC) or the synovial fluid mononuclear cells (SFMC) with one of said ECGF Peptides or Epitopes; and (c) measuring T-Cell proliferation in vitro or secretion of IFN-γ into cell culture supernatants as a test for T-cell reactivity.

15. The method of claim 14, wherein the subject suffers from antibiotic-refractive Lyme arthritis.

16. The method of claim 15, wherein the subject suffers from antibiotic-responsive Lyme arthritis.

17. A method for determining whether a subject, suffering from chronic inflammatory arthritis, contains a B-cell response to ECGF resulting in the production of autoantibodies found in serum or synovial fluid against ECGF, comprising the steps of: (a) providing an isolated antigen, wherein said antigen is ECGF, ECGF peptide or ECGF epitope or a functionally equivalent portion, fragment, analog or derivative thereof; (b) providing a biological sample from a subject; (c) conducting an immunoassay on said sample utilizing said antigen; wherein said immunoassay detects the presence of antibodies that recognize said ECGF; and (d) determining that the subject contains an antibody against said antigen if the results of the immunoassay indicate that an antibody that recognizes said antigen is present in said sample.

18. The method of claim 17, wherein the subject suffers from antibiotic-refractory Lyme arthritis.

19. The method of claim 18, wherein the subject suffers from antibiotic-responsive Lyme arthritis.

20. The method of claim 18, wherein the immunoassay is an enzyme-linked immunosorbent assay (ELISA).

21. (canceled)

22. (canceled)

23. (canceled)

24. (canceled)

25. (canceled)

26. (canceled)


SEQUENCE LISTING (8 sequences)

SEQ ID NO: 1 - protein, 16 aa, Homo sapiens
LGRFERMLAAQGVDPG

SEQ ID NO: 2 - protein, 20 aa, Homo sapiens
ADIRGFVAAVVNGSAQGAQI

SEQ ID NO: 3 - protein, 15 aa, Homo sapiens
DKVSLVLAPALAACG

SEQ ID NO: 4 - protein, 15 aa, Homo sapiens
SKKLVEGLSALVVDV

SEQ ID NO: 5 - protein, 22 aa, Homo sapiens
KTLVGVGASLGLRVAAALTAMD

SEQ ID NO: 6 - protein, 15 aa, Homo sapiens
LRDLVTTLGGALLWL

SEQ ID NO: 7 - protein, 16 aa, Homo sapiens
GTVELVRALPLALVLH

SEQ ID NO: 8 - protein, 482 aa, Homo sapiens
MAALMTPGTGAPPAPGDFSGEGSQGLPDPSPEPKQLPELIRMKRDGGRLSEADIRGFVAAVVNGSAQGAQIGAMLMAIRLRGMDLEETSVLTQALAQSGQQLEWPEAWRQQLVDKHSTGGVGDKVSLVLAPALAACGCKVPMISGRGLGHTGGTLDKLESIPGFNVIQSPEQMQVLLDQAGCCIVGQSEQLVPADGILYAARDVTATVDSLPLITASILSKKLVEGLSALVVDVKFGGAAVFPNQEQARELAKTLVGVGASLGLRVAAALTAMDKPLGRCVGHALEVEEALLCMDGAGPPDLRDLVTTLGGALLWLSGHAGTQAQGAARVAAALDDGSALGRFERMLAAQGVDPGLARALCSGSPAERRQLLPRAREQEELLAPADGTVELVRALPLALVLHELGAGRSRAGEPLRLGVGAELLVDVGQRLRRGTPWLRVHRDGPALSGPQSRALQEALVLSDRAPFAAPSPFAELVLPPQQ